(19) United States
(12) Patent Application Publication
(10) Pub. No.: US 2020/0276458 A1
(43) Pub. Date: Sep. 3, 2020

(54) SURFACTANT-FREE FOAM FORMULATIONS

(71) Applicant: NEUBOURG SKIN CARE GMBH & CO. KG, Greven (DE)

(72) Inventor: Rolf DANIELS, Rottenburg a.N. (DE)

(21) Appl. No.: 16/594,233

(22) Filed: Oct. 7, 2019

Related U.S. Application Data
(63) Continuation of application No. 15/585,955, filed on May 3, 2017, now abandoned.
Application No. 15/585,955 is a continuation of application No. 12/599,561, filed on Jan. 7, 2010, now abandoned.

(30) Foreign Application Priority Data
May 10, 2007 (EP) 07107950.3

(51) Int. Cl. A61Q 19/00; A61K 47/14; A61K 8/73; A61K 9/12; A61K 8/04; A61K 8/06; A61K 8/27; A61K 8/29

(52) U.S. Cl. CPC A61Q 19/00; A61K 47/14; A61K 8/731; A61K 9/124; A61K 2800/33; A61K 8/06; A61K 8/062; A61K 8/27; A61K 8/29; A61K 8/046

(57) ABSTRACT

The invention relates to a foam formulation comprising an essentially emulsifier-free emulsion of the oil in water type, comprising an oil phase and a water phase.

### TECHNICAL FIELD OF THE INVENTION

The present invention concerns cosmetic and dermatologic foam formulations based on emulsions of the oil-in-water type which are free or essentially free of conventional emulsifiers. In particular, the present invention relates to the use of Pickering emulsions for the manufacture of foams.

### BACKGROUND OF THE INVENTION

### 1. Emulsions

The term “emulsion” generally relates to heterogenous systems consisting of two liquids that are not miscible or are only miscible to a limited extent which are typically designated as phases. In an emulsion, one of both liquids is dispersed in the other liquid in the form of fine droplets.

In case that the two liquids are water and oil and the oil droplets are finely dispersed in water, the emulsion is an oil-in-water emulsion (O/W emulsion, e.g. milk). The basic character of an O/W emulsion is defined by the water. In case of a water-in-oil emulsion (W/O emulsion, e.g. butter), the opposite principle applies wherein the basic character is here defined by the oil.

In order to obtain a durable dispersion of a liquid in another liquid, emulsions in a conventional sense require the addition of a surface active agent (emulsifier). Emulsifiers have an amphiphile molecular structure consisting of a polar (hydrophile) and a non-polar (lipophile) part of the molecule which are separated from each other in space. In simple emulsions, finely dispersed droplets enclosed by an emulsifier shell of the one phase are present in the second phase (water droplets in W/O or lipid vesicles in O/W emulsions). Emulsifiers reduce the surface tension between the phases because they are arranged in the boundary surface between the two liquids. They form surface films at the boundary of the oil/water phases which countervails an irreversible joining of the droplets. For stabilizing emulsions mixtures of emulsifiers are often used.

The term “emulsifier” or “conventional emulsifier”, respectively, is known in the art. Conventional emulsifiers are described, e.g., in the publications: Pflegekosmetik, 4th edition, Wissenschaftliche Verlagsgesellschaft mbH Stuttgart, pages 151 to 159 and Fiedler Lexikon der Hilfsstoffe, 5th edition, Editio Cantor Verlag, Aulendorf, pages 97 to 121.

Conventional emulsifiers can be classified depending on their hydrophile part of the molecule into ionic (anionic, cationic and amphoteric) and non-ionic ones:
The best known example of an anionic emulsifier is believed to be soap which is the conventional name for the water-soluble sodium or potassium salts of saturated and non-saturated higher fatty acids.
Important members of cationic emulsifiers are the quaternary ammonium compounds.
The hydrophilic part of the molecule of non-ionic emulsifiers often consists of glycerol, polyglycerol, sorbitanes, carbohydrates or polyoxyethylene glycols, respectively, and is most often connected to the lipophilic part of the molecule by means of ester and ether bonds. The latter consists typically of fatty alcohols, fatty acids or iso-fatty acids.

By variation of the structure and the size of the polar and the non-polar part of the molecule, lipophilicity and hydrophilicity of emulsifiers can be modified to a large extent.

The correct choice of emulsifiers is decisive for the stability of an emulsion. In this respect, the characteristics of all compounds contained in the system need to be considered. For example, in case of skin care emulsions, polar oil components such as e.g. UV filters may lead to instabilities. Apart from emulsifiers, other stabilizers are, therefore, additionally used, which, e.g., increase the viscosity of the emulsion and/or act as protective colloid.

Emulsions represent an important type of product in the field of cosmetic and/or dermatologic preparations which is used in different application fields. Therefore, a variety of products—such as lotions and creams—are available for skin care, especially for relubricating dry skin. The aim of skin care is to compensate for the loss of lipid and water caused by daily washing. In addition, such skin care products should protect from environmental stress—in particular from sun and wind, and should delay skin ageing.

Cosmetic emulsions are also used as deodorants. Such formulations are used for eliminating the adore of the body that is formed when fresh sweat that as such is free of odour is decomposed by microorganisms.

Emulsions in the form of cleaning emulsions are also used for cleaning of the skin and skin adnexa. They are most often used for the cleaning of the face and especially for removing decorative cosmetic. Such cleaning emulsions have the advantage—in contrast to other cleaning preparations such as soap—to be especially mild on skin since they may contain in the lipophilic phase nurturing oils and/or non-polar active agents—such as, e.g., vitamin E.

### 2. Emulsions Free of Emulsifiers

Emulsifier-free emulsions are a special form of an emulsion. These emulsions are free of emulsifiers in a narrower sense, i.e. free of amphiphilic compounds having a low molecular weight (molecular weight of <5000) that in higher concentrations form micelles and/or other liquid crystalline aggregates. The term “emulsifier” is used here in the sense as usual in the art. The IUPAC defines the properties of an emulsifier as follows: Emulsifiers are surface-active substances. They are preferably arranged in the boundary surface between oil phase and water phase and, therefore, reduce the surface tension. In low concentration, emulsifiers facilitate the formation of an emulsion. In addition, these substances may increase the stability of emulsions in that they reduce the rate of aggregation and/or coalescence.

For stabilizing pharmaceutical and cosmetic emulsions, so-called true emulsifiers are predominantly used, i.e. conventional emulsifiers in the sense of the present description that according to their structure and their physical-chemical behaviour belong to the class of tensides. They are characterized in an amphiphilic structure and the capability for micelle association.

The term “emulsifier-free” is established in the art. According to a definition of the term agreed upon by an interdisciplinary consensus of pharmacists, dermatologists and other experts of the Society of Dermatopharmacie

(http://www.dermotopics.de/german/ausgabe_1_03_d/emulgatorfrei_1_2003_d.htm) a formulation may be defined as “emulsifier-free” when it is stabilized by means of surface active macromolecules (having a molecular weight over 5000) instead of emulsifiers in a narrower sense (conventional emulsifiers).

### 3. Pickering Emulsion

A special form of emulsifier-free emulsions is the Pickering emulsion. Pickering emulsions (emulsions stabilized by solids) are stabilized by means of finely divided solid particles and allow for the substantial abdication of conventional emulsifiers.

In Pickering emulsions, the solids accumulate in the oil/water boundary surface in the form of a layer whereby the joining of the dispersed phases is prevented. In this respect, the wetting properties of the solid particles, which should be wettable by both the hydrophilic as well as the lipophilic phases, are of special importance.

### 4. Foam Formulations

A special application form of cosmetic and/or dermatologic emulsions is the application as foams. Foam formulations have the advantage that they can easily be distributed on the skin. The foamy consistency is experienced as comfortable and the products normally leave a good skin feeling. In particular, the physical structure of the foam acts positively on the protective action of the skin. Foams are complicated physical structures that require a special balance of the components constituting the foam. In general, foams are obtained by spraying a formulation of an emulsion or an aqueous tenside (stabilizer) solution. For example, an emulsion containing propellant is dispensed from a pressurized container (such systems are also described in literature and patent literature as aerosol foams). In this case, the pressurized mixture of emulsion and propellant expands and forms small foam bubbles. In particular, the dispersed oil phase in which the oil-soluble gas is dissolved expands. However, foams can also be formed by means of other systems such as, for example, pump sprays.

Upon application, balanced foam formulations have a stable polydisperse structure of two or more phases that forms on the skin a network structure that is comparable to a membrane. Such network structures have the advantage that they develop a protective action, for example against contact with water, however, allow for the unhindered gas exchange with the environment. In such foams, there is practically no obstacle for the perspiratio insensibiles and no corresponding heat build-up. Thus, the positive properties of a protective and nurturing action is combined with an unchanged perspiration.

Foam formulations known so far contain conventional tensile/emulsifiers that serve for the stabilization of the emulsion and for the resulting foam stability.

Conventional emulsifiers or tensides, respectively, are, however, repeatedly identified as causing irritations in the use of skin care products, such as e.g. a dysfunction of the skin barrier or Mallorca acne. The addition of suitable stabilizers can, however, not be completely abdicated, since dispersed systems as described above, such as e.g. emulsions, are thermodynamically unstable.

The Pickering emulsions described above are an option to avoid emulsifiers. In EP 1 352 639 A1 or DE 101 62 840, respectively, Pickering emulsions are described which are, however, used as emulsions in the form of lotions, creams and gels.

In WO 2004/017930, further Pickering emulsions are described which are characterized especially in a low viscosity and, therefore, are suitable for dermatologic cloths. Such thin fluid Pickering emulsions even can be sprayed under formation of haze.

However, none of the above-described documents specifies foam formulations on the basis of Pickering emulsions free of emulsifiers.

### SUMMARY OF THE INVENTION

The applicant has found that Pickering emulsions are suitable as a basis for foam formulations. Thus, the positive properties of foam formulations are combined with those of Pickering emulsions. In particular, foam formulations can be produced without conventional emulsifiers or with very low amounts of conventional emulsifiers combining the positive properties of the foam, i.e. the physical structure and convenient application, with the good skin compatibility. This combination of properties makes foam formulations especially suitable for cosmetic and dermatologic formulations for sensitive types of skin. Thus, skin compatibility and convenience of application is combined advantageously with each other.

In this respect it has first of all to be considered that it is not obligatory that foaming of Pickering emulsions stabilized by means of solids leads to stable foam products. Foams are obtained, as already mentioned, e.g. by incorporating propellants into O/W emulsion systems. In case that the propellant dissolved in the dispersed oil phase evaporates upon foaming, a foam is formed (dispersion of gas in liquid). Foaming or expanding, respectively of the propellant dissolved in the dispersed oil phase leads to a dilatation of the dispersed oil phase. In this respect it is surprising that the network of solids at the boundary surface is able to withstand the dilatation stress and that upon foaming no breaking of the formulation occurs.

Thus, the invention relates to foam formulations comprising a Pickering emulsion of the type oil in water comprising an oil phase and an aqueous phase and optionally propellant. In particular, the invention relates to foam formulations containing not more than 0.5 weight percent of conventional emulsifiers, preferably not more than 0.3 weight percent, more preferably not more than 0.1 weight percent and even more preferably being free from conventional emulsifiers. The data is based on the respective total weight of the emulsion without propellant.

Furthermore, the invention relates to the use of Pickering emulsions, in particular, Pickering emulsions being substantially free from or being free from conventional emulsifiers, for the manufacture of foam formulations.

In addition, the invention relates to the use of foam formulations on the basis of Pickering emulsions as carrier for active agents, as skin care agent, as skin cleaning agent or as sunscreen. The foam formulation can, therefore, be employed as cosmetic, medical product or pharmaceutical composition.

The invention further relates to a method for manufacture of foam formulations based on Pickering emulsions. The method comprises the steps:
a) Producing a Pickering emulsion of the oil in water type,
b) Filling the Pickering emulsion and propellant into a pressurized container or
c) Filling the Pickering emulsion into a container other than a pressurized container which produces a foam upon dispensing of the Pickering emulsion.

### DETAILED DESCRIPTION OF THE INVENTION

### 1. Definitions

According to the present invention, foam formulations are formulations, especially emulsions, that are evidently adapted for the formation of a foam. In particular, the formulations may be either filled together with a propellant in a pressurized container or may be filled without propellant in a container other than a pressurized container that allows for the formation of a foam upon dispensing of the formulation/emulsion. For example, pump spray containers may be used.

According to the present invention, essentially emulsifier-free emulsions are such emulsions that do not contain more than 0.5 weight percent of conventional emulsifiers, preferably not more than 0.3 weight percent, more preferably not more than 0.1 weight percent. According to the invention, emulsifier-free emulsions are such emulsions that do not contain conventional emulsifiers.

According to the present invention, a Pickering emulsion is an emulsion that is stabilized by means of solids. In particular, the invention especially relates to an emulsion stabilized by means of solids of the oil in water type (O/W emulsion). In particular, the emulsion stabilized by means of solids according to the invention comprises not more than 0.5 weight percent of conventional emulsifiers, preferably not more than 0.3 weight percent, more preferably not more than 0.1 weight percent. Pickering emulsions according to the invention being free from conventional emulsifiers are especially preferred.

According to a further aspect, conventional emulsifiers are according to the present invention anionic, cationic, amphoteric and non-ionic tensides. Typical members of anionic tensides are neutralized branched and/or non-branched, saturated or unsaturated fatty acids having a chain length of 10 to 40 carbon atoms. Typical members of cationic tensides are ammonium compounds. Typical members of non-ionic tensides have a hydrophilic part of the molecule, such as glycerol, polyglycerol, sorbitane, carbohydrates or polyoxyethylene glycols, respectively, that is bonded by means of ester and/or ether bonds to the lipophilic part of the molecule which typically consists of fatty alcohols, fatty acids or iso-fatty acids. For example, polyethoxylated fatty acid esters having a chain length of 10 to 40 carbon atoms and a degree of ethoxylation of 5 to 100 belong to this group. Furthermore, saturated and/or unsaturated, branched and/or non-branched fatty alcohols having a chain length of 10 to 40 carbon atoms belong to the group of non-ionic emulsifiers. Conventional emulsifiers are often used in combination. Conventional emulsifiers in the sense of the present description are specified in the publications: Pflegekosmetik, 4th edition, Wissenschaftliche Verlagsgesellschaft mbH Stuttgart, pages 151 to 159 and Fiedler Lexikon der Hilfsstoffe, 5th edition, Editio Cantor Verlag, Aulendorf, pages 97 to 121.

According to a further aspect of the invention, conventional emulsifiers in the sense of the present invention are all amphiphilic compounds having a molecular weight of <5000 that in higher concentration may form micelles and/or other liquid crystalline aggregates. According to an even further aspect, conventional emulsifiers are all surface active substances that are not present as solid or polymer, especially under conventional temperatures of storage and application, such as e.g. room temperature.

According to the invention, a solid emulsifier is a particulate substance that is wettable by both lipophilic as well as hydrophilic liquids. In this respect, inorganic or organic solids are possible. Furthermore, the particles may be untreated or coated. The particle size is preferably between 1 nm and 200 nm, more preferably between 5 nm and 100 nm.

### 2. Composition of the Pickering Emulsion

Solid Emulsifiers:

Suitable solid emulsifiers are particulate inorganic or organic solids that are wettable by both lipophilic as well as hydrophilic liquids. Suitable members are e.g. titanium dioxide, especially coated titanium dioxide (e.g. obtainable from Merck KGaA under the designation Eusolex® T-2000), tin oxide (e.g. obtainable from BASF AG under the design Z-Cote Max), silicon dioxide, especially highly dispersed silicon dioxide, Fe₂O₃, veegum, bentonit and ethyl cellulose. Furthermore, aluminum oxide, nanoparticulate precipitated calcium carbonate, coal, magnesium oxide, magnesium trisilicate, crystalline fatty alcohols and fatty acids, polymer lattices, e.g. polystyrenes or polymethacrylates and polymer pseudo lattices may be used. In addition, mixtures of the above-mentioned solid emulsifiers may be used. Coated titanium dioxide or tin oxide is preferred.

The inventive emulsions contain more than 1 weight percent solid emulsifier, preferably more than 2 weight percent solid emulsifier, especially 2 to 7 weight percent solid emulsifier. In an especially preferred embodiment, the inventive emulsions contain 3 to 4 weight percent solid emulsifier. The data respectively refers to the total weight of the emulsion without propellant.

Oil Phase:

Suitable components that may form the oil phase may be selected from polar oils and unpolar oils or mixtures thereof.

The oil phase of the inventive formulations are advantageously selected from the group of lecithins and of fatty acid triglycerides, from the group of propylene glycols or butylene glycols, fatty acid esters, from the group of natural waxes of animal or plant origin, from the group of ester oils, from the group of dialkyl ethers and dialkyl carbonates, from the group of branched and non-branched hydrocarbons and waxes as well as from the group of cyclic and linear silicon oils.

Especially preferred are triglycerides, in particular caprylic acid/caprinic acid triglyceride obtainable under the designation Miglyol 812 of the company Sasol and mixtures thereof with further oil and wax components.

Furthermore, especially preferred are triglycerides, in particular caprylic acid/caprinic acid triglyceride obtainable under the designation Miglyol 812 of the company Sasol/Myritol 312 of the company Cognis.

The inventive emulsions preferably contain from 10 to 50 weight percent oil phase, especially preferably 25 to 35 weight percent oil phase. The data respectively refers to the total weight of the emulsion without propellant.

Aqueous Phase:

The aqueous phase can contain cosmetic adjuvants, e.g. lower alcohols (e.g. ethanol, isopropanol), lower dioles or polyoles as well as ethers thereof (e.g. propylene glycol, glycerole, butylene glycol, hexylene glycol and ethylene glycol), foam stabilizers and thickening agents.

Suitable thickening agents are polymeric thickening agents that are partly soluble in water or are at least dispersible in water and form in aqueous systems gels or viscous solutions. They increase the viscosity of the water in that they either bind water molecules (hydratation) or, on the other hand, include and encapsulate the water into their intertwined macromolecules wherein movability of the water is decreased.

Suitable Polymers are:

modified natural materials, such as cellulose ether (e.g. hydroxypropyl cellulose ether, hydroxyethyl cellulose and hydroxypropylmethyl cellulose ether);

natural compounds, such as e.g. agar-agar, carrageen, polyoses, starch, dextrins, gelatine, casein;

synthetic compounds, such as e.g. vinyl polymers, polyether, polyimines, polyamides and derivates of polyacrylic acid; and

inorganic compounds, such as e.g. polysilicic acid and clay minerals.

Preferably, a cellulose ether is contained. Hydroxypropylmethyl cellulose is especially preferred.

The inventive emulsions preferably contain from 0.2 to 1.5 weight percent thickening agent (based on the dry weight of the thickening agent and the total weight of the emulsion without propellant). Especially preferred are 0.5 to 0.8 weight percent thickening agent.

Active Agents:

The contained active agent may be selected from all active agents and mixtures thereof that can be applied to the surface of the skin. The active agent can act cosmetically or pharmaceutically. Accordingly, cosmetic or dermatologic (to be employed as medical product or pharmaceutical composition) foam formulations are obtained. Furthermore, the formulation may be employed for protecting the skin against environmental influences. The active agent can be completely of plant origin or can be synthetic. The group of active agents may overlap with other groups of ingredients, such as e.g. the oil component, the thickening agents or the solid emulsifiers. For example, some oil components also may act as active agents, such as e.g. oils having polyunsaturated fatty acids or solid emulsifiers, such as e.g. particulate titanium dioxide that may serve as UV-filter. Depending on the characteristics, the substances are to be classified into several groups.

Active agents of the inventive formulations are advantageously selected from the group of substances having moisturizing and barrier strengthening properties, such as e.g. hydroviton, an emulation of NMF, pyrrolidone carbonic acid and salts thereof, lactic acid and salts thereof, glycerol, sorbitol, propylene glycol and urea, substances of the group of proteins and protein hydrolysates, such as e.g. collagen, elastin as well as silk protein, substances of the group of glycose aminoglucanes, such as e.g. hyaluronic acid, of the group of carbohydrates, such as e.g. pentavitin that corresponds in its composition to the carbohydrate mixture of the human subcorneus layer and the group of lipids and lipid precursors such as for example ceramides. Further advantageous active agents in the sense of the present invention may be selected from the group of vitamins, such as e.g. panthenol, niacin, α-tocopherol and its esters, vitamin A as well as vitamin C. Moreover, active agents selected from the group of antioxidants e.g. galates and polyphenoles may be used. Urea, hyaluronic acid and pentavitin are preferred substances.

It is further preferred that substances having skin soothing and regenerative action are employed as active agents, such as e.g. panthenol, bisabolol and phytosteroles.

Advantageous active agents in the sense of the present invention are also plants and plant extracts. These are e.g. algae, aloe, arnica, barber's rash, comfrey, birch, nettle, calendula, oak, ivy, witch hazel, henna, hop, camomile, ruscus, peppermint, marigold, rosemary, sage, green tea, tea tree, horsetail, thyme and walnut as well as extracts thereof.

The inventive formulations may further contain as active agents antimycotics and antiseptics/disinfectants of synthetic or natural origin.

Further active agents are glycocorticoides, antibiotics, analgetics, antiphlogistics, antirheumatics, antiallergics, antiparasitics, antipruriginosics, antipsoriatics, retinoids, local anaesthetics, therapeutic agents for veins, ceratolytics, hyperemic substances, coronary therapeutic agents (nitrates/nitro-compounds), virus statics, cytostatics, hormones, agents promoting wound healing, e.g. growth factors, enzyme preparations and insecticides.

Further Components of the Pickering Emulsion:

The formulations may contain colouring agents, pearlescent pigments, fragrances/perfumes, sunscreen filter substances, preservatives, complex formers, antioxidants and repellent agents.

The above list of individual components of the Pickering emulsion should be considered such that individual exemplified components may be classified into several groups because of its different properties.

Propellants:

Suitable propellants are e.g. N₂O, propane, butane and i-butane. The completed foam formulation contains 5 to 15 weight percent of propellant, preferably about 10 weight percent.

### 3. Method of Manufacture

The foam formulations according to the invention are prepared by providing an emulsion or Pickering emulsion, respectively, of the oil-in-water type and filling the emulsion or Pickering emulsion, respectively and optionally propellant into an optionally pressurized container. As an alternative to propellant and pressurized container, the Pickering emulsion may also be filled into a different container that is suitable to dispense the Pickering emulsion as a foam even in the absence of propellant.

In particular, the Pickering emulsion is prepared in the following steps:
1. Providing a liquid oil phase,
2. Suspending the solid emulsifier(s) in the oil phase in order to obtain a suspension of solid emulsifier,
3. Providing an aqueous phase,
4. Homogenizing the aqueous phase together with the suspension of solid emulsifier in order to obtain a Pickering emulsion.

Preferably, the oil phase and the aqueous phase are homogenized at a temperature of from 50 to 90° C., preferably from 50 to 70° C. and especially preferably at about 60° C.

In case the Pickering emulsion comprises a thickening agent, the following further steps are advantageously added:
5. Providing an aqueous solution of thickening agent,
6. Mixing the solution of thickening agent with the Pickering emulsion.

Preferably, 10 weight percent propellant are added to the Pickering emulsion for producing the foam formulation.

### 4. Applications

The foam formulations of the present invention can be employed for all cosmetic and dermatologic (as a medical product or pharmaceutical composition) purposes. For example, the formulations may be employed as skin care agent or skin cleaning agent. Further, they may be used as carriers for active agents and may be employed in the medical dermatologic field. In particular, the formulations may be employed as sunscreen. Many of the solid emulsifiers such as for example titanium dioxide are effective UVA and UVB filters.

### 5. Examples

### 5.1. Example 1

Composition of the Primary Pickering Emulsion:

10.00 g Eusolex T 2000 of the company Merck KGaA (Alumina/Simethicone coated titanium dioxide)

95.00 g Miglyol 812 of the company Sasol (caprylic acid/caprinic acid triglyceride)

95.00 g water

Composition of the Foam Formulation:

60.00 g primary Pickering emulsion

30.00 g aqueous solution of hypromellose (2%)

10.00 g propellant

Manufacture of the Primary Pickering Emulsion:

Eusolex T2000 is weighted and is transferred into an laboratory homogenizer and is mixed with the Miglyol 812 heated to 60° C. within one minute and is homogenized within 5 minutes. The water is heated to 60° C. and is added at 1000 rpm within one minute. The mixture is homogenized at 3000 rpm for 5 minutes.

Manufacture of the Foam Formulation:

30.00 g solution of hypromellose are added to 60.00 g of the primary Pickering emulsion under stirring. The Pickering emulsion containing hypromellose is then loaded with 10.00 g propellant in aluminum monoblock cans.

Formation of Foam:

A stable cream-like foam having fine bubbles is formed upon dispensing of the foam formulation from the pressurized container by means of a suitable valve having a foam applicator attached. The structure of the cream-like foam is maintained for a duration sufficient for uniformly distributing the foam on the skin.

### 5.2. Example 2

Composition of the Primary Pickering Emulsion:

10.00 g Eusolex T 2000 of the company Merck KGaA (Alumina/Simethicone coated titanium dioxide)

30.00 g Cetiol V of the company Cognis (Decyloleat)

30.00 g Myritol 318 of the company Cognis (caprylic acid/caprinic acid triglyceride)

30.00 g almond oil

100.00 g water

Composition of the Xanthan Gum Solution (1%):

1.0 g xanthan gum

99.00 g water

Composition of the Foam Formulation:

60.00 g primary Pickering emulsion

30.00 g aqueous xanthan gum solution (1%)

10.00 g propellant

Manufacture of the Primary Pickering Emulsion:

Eusolex T 2000 is weighted and is transferred into a laboratory homogenizer and is mixed with the mixture of Cetiol V, Myritol 318 and almond oil heated to 60° C. within one minute and is homogenized within 5 minutes. The water is heated to 60° C. and is added at 1000 rpm within one minute. The mixture is homogenized at 3000 rpm for 5 minutes.

Manufacture of the Foam Formulation:

30.00 g xanthan gum solution are added to 60.00 g of the primary Pickering emulsion under stirring. The Pickering emulsion containing the xanthan gum solution is loaded with 10.00 g propellant in an aluminum monoblock can.

Foam Formation:

A stable cream-like foam having fine bubbles is formed upon dispensing the foam formulation from the pressurized container by means of a suitable valve having a foam applicator attached. The structure of the cream-like foam is maintained for a duration sufficient for uniformly distributing the foam on the skin.

### 5.3. Example 3

Composition of the Primary Pickering Emulsion:

10.00 g ZCote of the company BASF (tin oxide)

20.00 g Myritol 318 of the company Cognis (caprylic acid/caprinic acid triglyceride)

10.00 g Cetiol V of the company Cognis (Decyloleat)

20.00 g Cetiol SN of the company Cognis (Cetylstearylisononnanoat)

20.00 g almond oil

120.00 g water

Composition of the Solution of Hypromellose (2%):

2.00 g hypromellose

5.00 g glycerol

5.00 g urea

88.00 g water

Composition of the Foam Formulation:

40.00 g primary Pickering emulsion

52.00 g aqueous solution of hypromellose (2%)

8.00 g propellant

Manufacture of the Primary Pickering Emulsion:

ZCote is weighted and is transferred into a laboratory homogenizer and is mixed with the mixture of Cetiol V, Myritol 318, Cetiol SN and almond oil within one minute and is homogenized within 5 minutes. The water is added at 1000 rpm within one minute. The mixture is homogenized for 5 minutes at 3000 rpm.

Manufacture of the Foam Formulation:

52.00 g solution of hypromellose are added to 40.00 g of the primary Pickering emulsion under stirring. The Pickering emulsion containing hypromellose is then loaded with 8.00 g propellant in an aluminum monoblock can.

Foam Formation:

A stable cream-like foam having fine bubbles is formed upon dispensing the foam formulation from the pressurized container by means of a suitable valve having a foam applicator attached. The structure of the cream-like foam is maintained for a duration that is sufficient for uniformly dispersing the foam on the skin.

1. Foam formulation comprising an essentially emulsifier-free emulsion of the oil in water type, comprising an oil phase and a water phase.

2. Foam formulation according to claim 1, wherein the emulsion does not contain more than 0.5 weight percent, preferably not more than 0.3 weight percent, more preferably not more than 0.1 weight percent of conventional emulsifiers.

3. Foam formulation according to claim 1, comprising an emulsifier-free emulsion that does not contain conventional emulsifiers.

4. Foam formulation according to claim 1, wherein the emulsion is a Pickering emulsion.

5. Foam formulation according to claim 4, wherein the Pickering emulsion comprises at least one particulate solid emulsifier selected from the group consisting of titan dioxide, silicon dioxide, Fe₂O₃, zinc oxide, veegum, bentonit and ethyl cellulose, aluminum oxide, nanoparticulate precipitated calcium carbonate, coal, magnesium oxide, magnesium trisilicate, crystalline fatty alcohols and fatty acids, polymer lattices such as polystyrene or polymethacrylate, and polymer-pseudolattices or mixtures thereof.

6. Foam formulation according to claim 4, wherein the Pickering emulsion comprises coated titanium dioxide or zinc oxide.

7. Foam formulation according to claim 1, wherein the oil phase comprises at least one triglyceride.

8. Foam formulation according to claim 7, wherein the triglyceride comprises caprylic acid/caprinic acid triglyceride.

9. Foam formulation according to claim 1, wherein the emulsion comprises at least one thickening agent.

10. Foam formulation according to claim 9, wherein the thickening agent comprises hydroxypropyl methylcellulose.

11. Foam formulation according to claim 1, wherein the emulsion contains at least one active agent.

12. Foam formulation according to claim 11, wherein the active agent is selected from the group consisting of hydroviton, pyrrolidone carbonic acid and salts thereof, lactic acid and salts thereof, glycerol, sorbitol, propylene glycol, urea, collagen, elastin, silk protein, hyaluronic acid, pentavitin, ceramid, panthenol, niacin, cx-tocopherol and esters thereof, vitamin A, vitamin C, galates, polyphenols, panthenol, bisabolol, phytosteroles, glycocorticoides, antibiotics, analgetics, antiphlogistics, antirheumatics, antiallergics, antiparasitics, antipruriginosics, antipsoriatics, retinoids, local anaesthetics, venous therapeutics, ceratolytics, hyperemisic compounds, coronary therapeutics (nitrates/nitro-compounds), virus statics, cytostatics, hormones, agents promoting wound healing, growth factors, enzyme preparations, insecticides and plant material such as plant extracts of algae, aloe, arnica, barber's rash, comfrey, birch, stinging nettle, calendula, oak, ivy, witch hazel, henna, hops, camomile, ruscus, peppermint, marigold, rosemary, sage, green tea, tea tree, horsetail, thyme, and walnut or mixtures thereof.

13. Use of a Pickering emulsion for the manufacture of a foam formulation.

14. Use according to claim 13 for the manufacture of a foam formulation.

15. Use of a foam formulation according to claim 1 as a carrier for an active agent.

16. Use of a foam formulation according to claim 1 as skincare agent.

17. Use of a foam formulation according to claim 1 as skin cleaning agent.

18. Use of a foam formulation according to claim 1 as sunscreen agent.

19. Use of a foam formulation according to claim 1 for the manufacture of a cosmetic, a medical product or a pharmaceutical composition.

20. Method for producing a foam formulation based on a Pickering emulsion comprising the steps:
a) Producing a Pickering emulsion of the oil in water type,
b) Filling the Pickering emulsion and propellant into a pressure vessel, or
c) Filling the Pickering emulsion into a different container as a pressure vessel that produces a foam upon dispensing of the Pickering emulsion.

21. Method according to claim 20 for producing a foam formulation.

22. Method according to claim 21, wherein producing of the Pickering emulsion comprises the steps:
1. Providing a liquid oil phase,
2. Suspending one or more solid emulsifiers in the oil phase in order to obtain a suspension of solid emulsifier,
3. Providing an aqueous phase,
4. Homogenizing the aqueous phase with the suspension of solid emulsifier in order to obtain a Pickering emulsion.

23. Method according to claim 22, wherein the oil phase and the aqueous phase are homogenized at a temperature between 50 and 90° C.

24. Method according to claim 22 or 23, wherein the Pickering emulsion comprises a thickening agent, further comprising the steps:
5. Providing an aqueous solution of thickening agent,
6. Mixing the solution of thickening agent with the Pickering emulsion.

25. Method according to claim 20, wherein the foam formulation contains 10 weight percent of propellant.
